(19) United States
(12) Patent Application Publication
(10) Pub. No.: US 2008/0249452 A1
(43) Pub. Date: Oct. 9, 2008

(54) Wound-Covering Material

(76) Inventors: Moriaki Tanaka, Ueda-shi (JP); Makoto Shimizu, Ueda-shi (JP)

Correspondence Address: BIRCH STEWART KOLASCH & BIRCH, PO BOX 747, FALLS CHURCH, VA, 22040-0747, US

(21) Appl. No.: 11/596,895

(22) PCT Filed: Jul. 8, 2005
(86) PCT No.: PCT/JP05/12698

§ 371 Date: Feb. 4, 2008

(30) Foreign Application Priority Data
Sep. 22, 2004 (JP) 2004-274797

(51) Int. Cl. A61F 13/00

(52) U.S. Cl. 602/44; 602/45; 602/48

(57) ABSTRACT

The present invention provides a new wound-covering material. The wound-covering material is produced by carbonizing a silk material at temperature of 500-1000° C., and a large number of microfine holes are formed in a surface of the silk carbonized body. By carbonizing at such low temperature, the silk carbonized body has flexibility of the silk material, a significant antibacterial property, a high water-absorbing property and a high water absorption rate, so that the silk carbonized body is suitable for the wound-covering material.

### FIELD OF TECHNOLOGY

The present invention relates to a wound-covering material.

### BACKGROUND TECHNOLOGY

Wound-covering materials for curing a raw area of skin caused by wounds, burn injuries, etc. should have enough compatibility with skin, enough flexibility for following deformation of skin and no invectiveness.

Conventionally, gauze made of cotton woven or unwoven cloth has been used for absorbing secretions come out from a wound and protecting the wound so as not to broaden the wound.

However, the cotton gauze adheres to regenerated skin, so the regenerate skin is peeled off together with the gauze when the gauze is removed from the wound in the treatment process.

Japanese Patent Gazette No. 11-70160 discloses a wound-covering material made of an amorphous film, whose main components are fibroin and sericin and whose degree of crystallization is less than 10%. The wound-covering material has enough flexibility and enough water absorption rate, further it is hard to adhere to skin.

### Patent Document: Japanese Patent Gazette No. 11-70160

### DISCLOSURE OF THE INVENTION

The inventors found that a silk carbonized material has a significant antibacterial property and that the silk carbonized body treated by activation treatment has a higher water absorption rate, so it can be used as a superior wound-covering material.

Namely, the wound-covering material of the present invention is produced by carbonizing a silk material, and a large number of microfine holes are formed in a surface of the silk carbonized body.

Preferably, the silk material is carbonized at temperature of 500-1000° C.

In the wound-covering material, the silk carbonized body may include 5-35 wt % of nitrogen elements.

In the wound-covering material, the microfine holes may be formed by activation treatment.

In another case, the microfine holes may be formed by irradiating microwaves to the silk carbonized body.

The wound-covering material may be formed like woven cloth, knitted fabric, unwoven cloth or yarns.

The wound-covering material, which is formed like woven cloth, knitted fabric or unwoven cloth, may include a wound therapeutic drug.

Further, the silk carbonized body may be broken into shatters.

### EFFECTS OF THE INVENTION

Since the wound-covering material of the present invention is carbonized at low temperature, the carbonized material has flexibility of the silk material, a significant antibacterial property, a high water-absorbing property and a high water absorption rate, so that the wound-covering material is suitable for regenerating skin and is capable of preventing skin from damaging and breaking.

### BRIEF DESCRIPTION OF THE DRAWINGS

FIG. 1 is a raman spectrum chart of a carbonized body, which was formed by carbonizing a coarse-grained silk at temperature of 2,000° C.

FIG. 2 is a raman spectrum chart of a carbonized body, which was formed by carbonizing a coarse-grained silk at temperature of 700° C.

FIG. 3 is a raman spectrum chart of a carbonized body, which was formed by carbonizing a coarse-grained silk at temperature of 1,000° C.

FIG. 4 is a raman spectrum chart of a carbonized body, which was formed by carbonizing a coarse-grained silk at temperature of 1,400° C.

FIG. 5 is a An FE-SEM photograph of a silk material carbonized at temperature of 700° C.

FIG. 6 is a An FE-SEM photograph of a silk material carbonized at temperature of 2,000° C.

FIG. 7 is a photograph of a burn injury after a lapse of one day and a drawing of a measuring point.

FIG. 8 is a graph showing wound area ratios (%) of every measuring day with respect to a wound area of the first wounding day.

FIG. 9 is skin tissues around the burn injury after a lapse of three days. (A·B): low power field tissue images (Bar=1 mm) of sections of HE-stained skin. Rectangular parts M1, M2 and M3 indicate parts, whose thicknesses were measured. (C·D): enlarged images of the burn injury of (A) and (B) (Bar=200 μm). Epidermis (Epi), dermis (Der), subcutaneous tissue (SC) and cutaneous muscle (CM) are observed.

FIG. 10 is a graph of thickness of skin around the burn injury after a lapse of three days.

FIG. 11 is a graph showing percentage of subcutaneous tissue with respect to entire skin after a lapse of three days.

FIG. 12 is images of skin tissues immediately under the burn injury after a lapse of three days. (A·B): low power field tissue images (Bar=1 mm) of sections of skin after a lapse of three days. Crusta (an arrow) of a CS(+) group, which was peeled from epidermis, is observed. (C·D): high power field images of the burn injury of (A) and (B) (Bar=100 μm). Neutrophil having segmented forms infiltrate immediately under crusta, but number of the CS(+) group was much greater.

FIG. 13 is photographs of blood vessels in the subcutaneous tissue after a lapse of three days. Blood vessels were invisible to the naked eyes in areas surrounded by arrows. In CS(+), it is difficult to observe blood vessels in a broad area near the burn injury. Bar=5 mm.

### PREFERRED EMBODIMENTS OF THE INVENTION

Silk carbonized bodies used in the present invention are produced by carbonizing of silk materials at relatively low temperature. In the following description, the silk materials are woven fabrics, knitted works, powders, cotton, yarns, etc. of domesticated silkworms or wild silkworms. One or a plurality of kinds of the silk materials are carbonized.

The silk materials are carbonized at temperature of 1400° C. or below, preferably 500-1000° C. A carbonizing atmosphere is an inert gas atmosphere, e.g., nitrogen gas atmosphere, argon gas atmosphere, or a vacuum atmosphere so as not to burn the silk materials to cinders.

The silk materials should be carbonized in stages without rapid carbonizing.

For example, the silk material is primary-carbonized in the inert gas atmosphere with low temperature rising rate of 100° C./hour or less, preferably 50° C./hour or less, until reaching a first temperature (e.g., 500° C.), then the first temperature is maintained for several hours for primary carbonizing. The silk material is once cooled until reaching the room temperature, then the silk material is secondary-carbonized in the same atmosphere with low temperature rising rate of 100° C./hour or less, preferably 50° C./hour or less, until reaching a second temperature (e.g., 700° C.) and the second temperature was maintained for several hours for secondary carbonizing. Then, the silk material is cooled and taken out from a furnace. Note that, the silk material may be secondary-carbonized, without cooling, after the primary carbonizing.

And, the carbonizing conditions are not limited to the above described examples, and they may be optionally changed on the basis of kinds of silk materials, functions of carbonized materials, etc.

By carbonizing the silk material in stages or carbonizing the same with the low temperature rising rate and at temperature of 1,000° C. or below, rapid decomposition of the protein high-order structure, in which crystalline forms and noncrystalline forms of a dozen of amino acids are combined, can be avoided, especially a large amount of nitrogen components are left so that the silk carbonized body has a lot of functions.

By carbonizing at low temperature of 500-1,000° C., the silk carbonized body is not graphitized and has plasticity (flexibility).

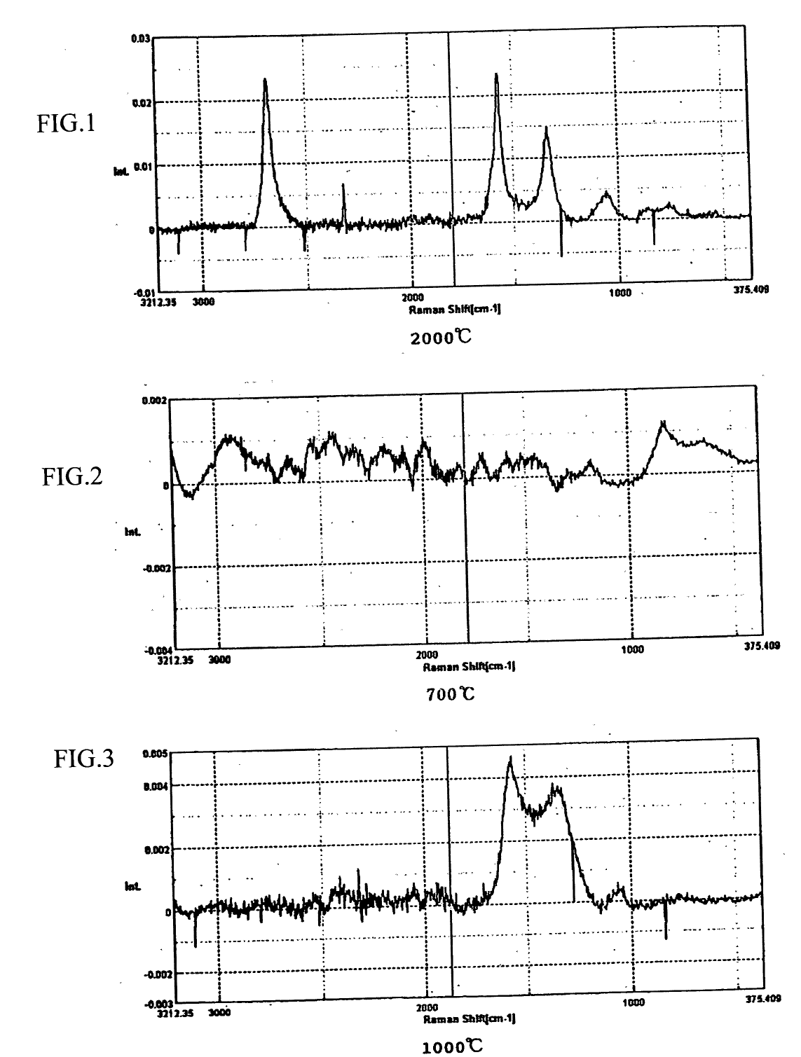

FIG. 1 is a raman spectrum chart of a carbonized body, which was produced by carbonizing a coarse-grained silk at temperature of 2,000° C. Peaks were observed at 2681cm⁻¹, 1570 cm⁻¹ and 1335 cm⁻¹, so the coarse-grained silk was graphitized.

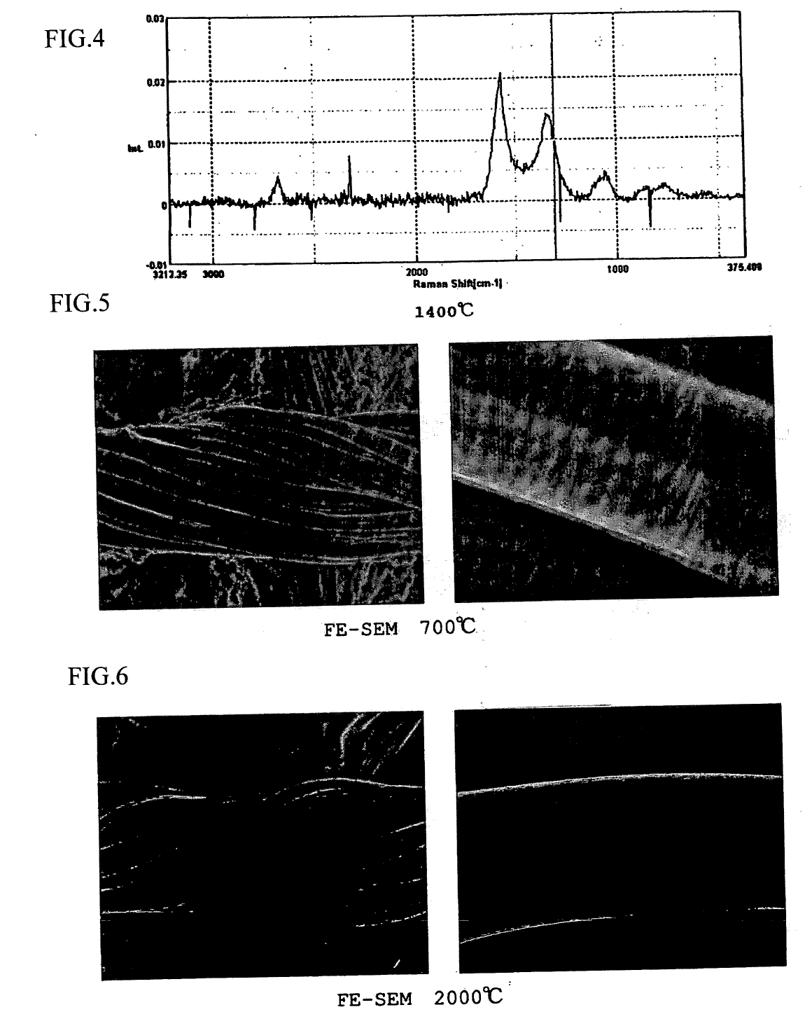

FIGS. 2-4 are raman spectrum charts of carbonized bodies, which were produced by respectively carbonizing coarse-grained silks at 700° C., 1,000° C. and 1,400° C. By carbonizing at 1,400° C., peaks were low but observed at the same three points. By carbonizing at 1,000° C. or below, the significant peaks were not observed, so the coarse-grained silk was little-graphitized.

TABLE 1

| | Elements | | | | | | | | | | | | |
|---|---|---|---|---|---|---|---|---|---|---|---|---|---|
| | C | N | O | Na | Mg | Al | Si | P | S | Cl | K | Ca | Fe |
| Wt % | 66.1 | 27.4 | 2.1 | 0.1 | 0.3 | 0.1 | 0.3 | 0.1 | 0.1 | 0.1 | 0.1 | 3.2 | 0.2 |

Table 1 shows results of elemental analysis (semiquantative analysis) of a carbonized product, which was a knitted work made of silk of domesticated silkworms and which was carbonized in a nitrogen atmosphere at 700° C., performed by an electron beam micro analyzer.

Measuring conditions were as follows: accelerating voltage was 15 kV; irradiating current was 1 μA; and probe diameter was 100 μm. Note that, values in the table indicate tendency of detected elements but they are not guaranteed values.

According to Table 1, a large amount of nitrogen components, i.e., 27.4 wt %, were remained. Further, other elements derived from amino acids were also remained.

TABLE 2

| | Elements | | | | | | | | | | | |
|---|---|---|---|---|---|---|---|---|---|---|---|---|
| | C | N | O | Na | Mg | Al | Si | P | S | Cl | K | Ca |
| Wt % | 74.6 | 15.7 | 5.1 | 0.3 | 0.3 | 0.1 | 0.7 | 0.1 | 0.2 | 0.2 | 0.1 | 2.7 |

Table 2 shows results of elemental analysis (semiquantative analysis) of a carbonized product, which was a knitted work made of silk of domesticated silkworms and which was carbonized in a nitrogen atmosphere at 1,400° C., performed by an electron beam micro analyzer.

Measuring conditions were as follows: accelerating voltage was 15 kV; irradiating current was 1 μA; and probe diameter was 100 μm. Note that, values in the table indicate tendency of detected elements but they are not guaranteed values.

According to Table 2, an amount of nitrogen components remained was reduced to 15.7 wt %.

TABLE 3

| | Elements | | | | | | | | | | |
|---|---|---|---|---|---|---|---|---|---|---|---|
| | C | N | O | Na | Mg | Al | Si | P | S | K | Ca |
| Wt % | 69.9 | 24.5 | 2.7 | 0.2 | 0.3 | 0 | 0.2 | 0.2 | 0.4 | 0.1 | 1.6 |

Table 3 shows results of elemental analysis (semiquantative analysis) of a carbonized product, which was an unwoven cloth made of silk of domesticated silkworms and which was carbonized in a nitrogen atmosphere at 700° C., performed by an electron beam micro analyzer.

Measuring conditions were as follows: accelerating voltage was 15 kV; irradiating current was 1 μA; and probe diameter was 100 μm. Note that, values in the table indicate tendency of detected elements but they are not guaranteed values.

According to Table 3, a large amount of nitrogen components, i.e., 24.5 wt %, were remained.

FIG. 5 is a FE-SEM photograph of a silk material carbonized at temperature of 700° C. A surface of the silk carbonized body is covered with thin films, which are burned residue derived from amino acids, e.g., nitrogen elements.

On the other hand, FIG. 6 is a FE-SEM photograph of a silk material carbonized at temperature of 2,000° C., but the surface of the silk carbonized body is clear and covered with no films.

TABLE 4

| | | Number of | | | |
|---|---|---|---|---|---|
| | Number | Bacteria in | Number of | Sterilization | Bacteriostatic |
| | of Inoculating | Untreated | Bacteria in | Activity | Activity |
| Bacteria | Bacteria | Cloth | Sample | Value | Value |
| Staph | 4.3 | 7.1 | 1.3 | 3.0 | 5.8 |
| Bacteria | 2.2E+04 | 1.2E+07 | 2.0E+01 | | |
| Klebsiella | 4.4 | 7.5 | 1.3 | 3.1 | 6.2 |
| Pneumoniae | 2.6E+04 | 3.0E+07 | 2.0E+01 | | |
| MRSA | 4.4 | 7.1 | 1.3 | 3.1 | 5.8 |
| | 2.6E+04 | 1.4E+07 | 2.0E+01 | | |
| Coli Bacteria | 4.3 | 7.5 | 1.3 | 3.0 | 6.2 |
| | 1.8E+04 | 2.9E+07 | 2.0E+01 | | |
| Pseudomonas | 4.2 | 7.0 | 1.3 | 2.9 | 5.7 |
| aeruginosa | 1.7E+04 | 1.1E+07 | 2.0E+01 | | |

Table 4 shows results of antibacterial tests of carbonized bodies, which were woven fabrics of domesticated silkworms and which were carbonized in a nitrogen atmosphere at temperature of 700° C.

The tests were JIS L 1902 quantative tests (unified tests).

Standard cotton cloth was used as the untreated cloth. “Number of Bacteria in Untreated Cloth” means number of bacteria, which have been inoculated and grown in the noncarbonized cloth.

For example, “2.2E+04” in the table is 2.2×10⁴, and the value “4.3” is a logarithm value thereof.

According to Table 4, bacteria considerably grew in the untreated cloth; on the other hand, all bacteria were considerably reduced in samples, i.e., carbonized cloth, so that we found that the carbonized body had an antibacterial property.

By carbonizing silk materials in a plurality of stages, carbonizing them at 1,400° C. or below and rising temperature with low temperature rising rate, a large amount of elements derived from amino acids, e.g., nitrogen elements, remained in the samples so that the samples could have the antibacterial property.

Next, an activation treatment is applied to the silk carbonized body so as to form a lot of microfine holes in a surface of the silk carbonized body and increase surface area thereof. By forming a lot of microfine holes in the surface, so that the water sorbability can be increased and moisture, blood or pus come out from a wound can be suitably absorbed. And, the antibacterial property can be further improved.

For example, the activation treatment is to expose the silk carbonized body to high-temperature steam, whose temperature is about 850° C. (1,000° C. or below); as a result of the activation treatment, a lot of microfine holes (diameters are from 0.1 nm to several dozen nm) are formed in the surface of the silk carbonized body.

Note that, the activation treatment may be performed by irradiating microwaves (e.g., frequency 2.45 GHz) to the silk carbonized body so as to form a lot of microfine holes. In case of irradiating microwaves, the silk carbonized body may be sandwiched between unglazed plates so as not to be incinerated.

In case of using the silk carbonized body as a wound-covering material, woven cloth, knitted fabric or unwoven cloth of the silk carbonized body may be used as gauze. Further, the silk carbonized body may be used in the form of yarns.

As described above, the silk carbonized body, which is produced by carbonizing the silk material at the low temperature, maintains flexibility and has excellent antibacterial property; the silk carbonized body can be used as the suitable wound-covering material. Further, the wound-covering material has excellent water sorbability, so that moisture, blood or pus come out from a wound can be suitably absorbed; the wound can be rapidly dried and cured.

A silk fiber is a bundle of many fibroin fibers, so spaces exist in the bundle; many of the bundles are woven and formed into, for example, cloth, so many spaces exist in the cloth; and many microfine holes are formed in a surface of each of the carbonized fibroin fibers; therefore, the wound-covering material has a three-dimensional structure including the spaces, so it has superior air permeability and moisture holding property and prevents regenerated skin from being peeled when the wound-covering material is peeled from a wound.

The wound-covering material may be formed like a gauze and used for covering a wound, further a wound therapeutic drug may be included in the wound-covering material formed like woven cloth, knitted work or unwoven cloth. For example, iodine, potassium iodine, etc., may be used as the wound therapeutic drug.

Further, the silk carbonized body may be broken into shatters having suitable sizes, and the shatters may be used as the wound-covering material solely or with the wound therapeutic drug. Namely, they may be used as a plaster.

### EXAMPLE 1

A silk material was heated in a nitrogen gas atmosphere until reaching first temperature (450° C.) with low temperature rising rate of 50° C./hour, then the material was carbonized at the first temperature for five hours as the primary carbonizing. Next, the carbonized material was once cooled until reaching the room temperature, then the material was reheated in the nitrogen gas atmosphere until reaching second temperature (700° C.) with low temperature rising rate of 50° C./hour, then the material was carbonized at the second temperature for five hours as the secondary carbonizing. Further, the carbonized material was cooled until reaching the room temperature, so that the silk carbonized body shown in FIG. 5 was produced.

The silk carbonized body was exposed to steam, whose temperature was 850° C., as the activation treatment, so that many microfine holes were formed in the surface of the silk carbonized body; the surface area of the silk carbonized body could be broadened about 1,000 times.

The silk carbonized bodies were formed into woven or unwoven cloth and used as the wound-covering material, so that the wound-covering material had superior flexibility and antibacterial property, and it was easily peelable from regenerated skin.

### EXAMPLE 2

Silk woven cloth, whose size was 9 cm×9 cm, was carbonized at temperature of 700° C., the carbonized body was sandwiched between unglazed plates and accommodated in a microwave oven, whose power was 500 W, then the carbonized body was irradiated by microwaves for six minutes.

Water absorption was measured under JIS L 1907, and the measured water absorption of the silk carbonized body before the microwave irradiation was 50%, but the measured water absorption of the silk carbonized body after the microwave irradiation was highly increased to 87%.

### EXAMPLE 3

Animal experiments with mice will be explained.

In the animal experiments, mice were burn-injured with three degree of burn, areas of the burn injuries were measured with the course of time. Since burn injuries are associated with edema, we thought that edema might be used as an index of estimating cure of the burn injuries; thus, we used percentages of ansarca with respect to subcutaneous tissues in and around the burn injuries as the index.

### 1. Process of The Experiments

### 1) Animal

27 ddy male mice of 7-10 weeks old were used.

### 2) Manner of Burn-Injuring

The mice were given anesthetic by sucking diethyl ether, hairs of backs of the mice were removed by a hair clipper and depilatory cream, and an electric soldering iron was pressed onto the backs for five seconds for making the burn injuries of three degree. The burn injuries were shot by a digital camera immediately after burn-injuring. Two mouse groups, each of which included seven mice, were formed; in one of the groups (hereinafter referred to as “CS(+) group”), ointment produced by mixing powders of the silk carbonized body (CS) of Example 1 with white petrolatum listed in the Japanese Pharamacopoeia (including 23 wt % of the powders of the silk carbonized body (CS)) was applied to the burn injuries of the mice; in the other group (hereinafter referred to as “CS(−) group”), the petrolatum including no powders of the silk carbonized body was applied to the burn injuries of the mice. The wounds were open wounds.

### 3) Estimation of the Injury Areas

The ointment and petrolatum were applied immediately after making the burn injuries and after lapse of one, three, six and eight days, and the burn injuries were shot by the digital cameras. Images shot by the digital camera were inputted to a computer, and areas of the burn injuries were measured by a free software “SCION”. Percentage (%) of the injury areas with respect to the original areas were calculated on each of the measuring days, differences depending on the absence or presence of the CS were statistically studied.

### 4) Histological Estimation

Two burn injuries were respectively made in backs of 10 mice, i.e., 20 burn injuries, by the electric soldering iron. Two mouse groups, each of which included five mice, were formed, and the burn injuries were treated as well as the above described treatment. After a lapse of three days, the mice, which had been given anesthetic by sucking diethyl ether, were sacrificed, then tissues of the wounds including peripheries were taken out. The tissues were soaked into 4% of paraformaldehyde and fixed, then paraffin cut pieces, whose thickness were 4 μm, were made and dyed by hematoxylin-eosin stain (HE stain) so as to produce tissue samples of skin sections. Picture of the samples were taken with an optical microscope, the tissues were observed, and thickness of skins and subcutaneous tissues were measured. Thickness of dermis was a distance from epidermis to dermo-adipous junction, thickness of the subcutaneous tissue was a distance from the dermo-adipous junction to cutaneous muscle, and the thickness of the skin was the total thickness of the both. Note that, if the tissue is diagonally cut, the thickness of the vertical section of the skin cannot be measured. Thus, percentages of the thickness of the subcutaneous tissues with respect to the total thickness of the entire skins were calculated so as to improve reliability of data. Measured points o were a position immediately below a center of each burn injury and three positions (M1, M2 and M3), which were located in the periphery of each burn injury and mutually separated about 500 μm. To obtain data of normal skins, similar samples were produced from male mice (three mice) of the same age in week and estimated the samples.

5) Observation of Blood Vessels around Wounds

Upon sacrificing the mice, skins were taken out, states of blood vessels in subcutaneous tissues were observed and shot by the camera.

### 6) Statistical Processing

A t-test was used for statistical processing, and risk rate of 5% or less was judged as significant. All of the data were indicated as “average+standard error”.

### 2. Results

1) Variations of Three-degree Burn Injuries with the Course of Time

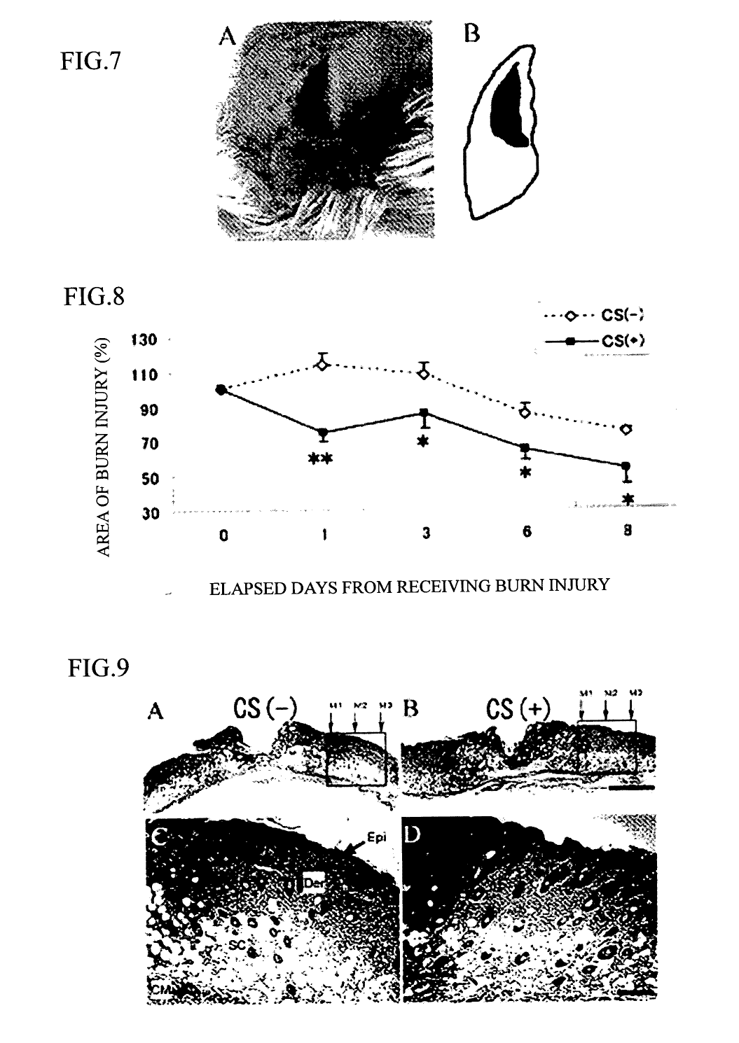

A photograph of the burn injury after a lapse of one day and a drawing of the measuring point are shown (see FIGS. 7A and B). In FIG. 7B, a blacken part is a part on which the electric soldering iron was applied, and the periphery is edema. Area of the burn injury includes the edema, which came up after making the burn injury. By comparing percentages (%) of the wound areas of the measuring days with respect to the initial area thereof (see FIG. 8), contractive tendency of the areas of the CS(+) group throughout the observation period was more significant than that of the CS(−) group. In the CS(−) group, the wound areas were broaden. for three days; in the CS(+) group, the wound areas were consistently reduced. This fact indicates that the edema of the CS(+) group were macroscopically lower-grade.

2) Skin Tissues around Burn Injury after a Lapse of Three Days

Low power field tissue images of sections of HE-stained skin (see FIGS. 9A and B) and high power field images around the burn injury are shown (see FIGS. 9C and D). The subcutaneous tissues around the burn injury of the CS(−) group had many parts not stained by eosin. This fact indicates that percentage of tissue spaces, blood vessels, lymph vessels was greater and the CS(−) group had much ansarca.

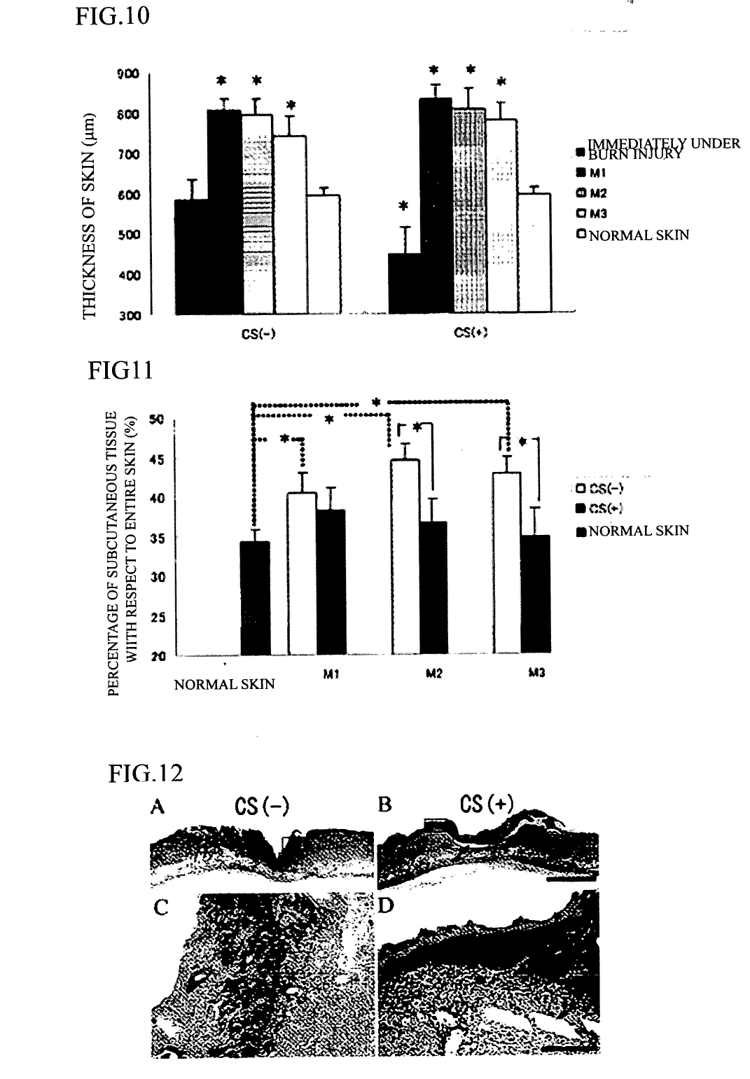

As to the thickness of the skins, the thickness of the measuring points close to the wounds were significantly thicker than that of the normal skins in the both groups, and significant differences were not observed between the CS(+) group and the CS(−) group (see FIG. 10). On the other hand, as to percentage of the subcutaneous tissue with respect to the entire skin, the percentages of the CS(−) group were significantly greater than that of the normal skins at all of the measuring points, but those of the CS(+) group were not significantly greater (see FIG. 11). At the measuring points M2 and M3, the percentages of the subcutaneous tissues of the CS(+) group were significantly smaller than those of the CS(−) group.

3) Skin Tissues Immediately under Burn Injury after a Lapse of Three Days

Thickness of the skins immediately under the burn injuries were compared with that of the normal skins; the thickness of the CS(+) group were significantly thinner, but the thickness of the CS(−) group were not significantly different (see FIG. 12). By precisely observing the tissue pieces, crusta peeled from epidermis of some samples were observed in the CS(+) group (see FIG. 12B). However, in the CS(−) group, no crusta peeled from epidermis was observed in this period (see FIG. 12A). In the CS(+) group, a lot of neutrophil having segmented forms, which were stained bluish-purple by hematoxylin, were infiltrated immediately under crusta (see FIGS. 12C and D). Therefore, it suggests that the CS accelerates ambulato of neutrophil and generation of crusta.

4) Blood Vessels in Subcutaneous Tissue after a Lapse of Three Days

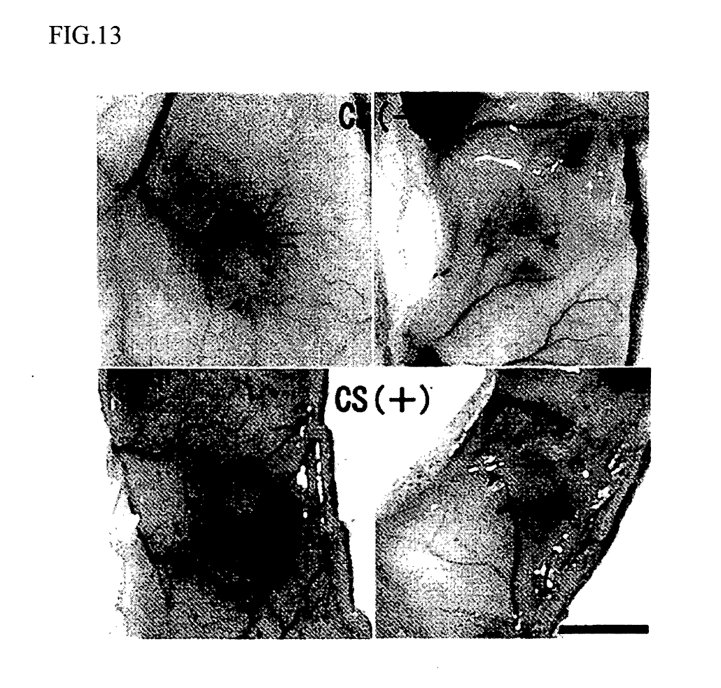

The burn injuries were observed from the subcutaneous tissue side immediately after the sacrifice so as to confirm if blood vessels were macroscopically observed or not. Typical examples are shown in FIG. 13. In the CS(+) group, it was difficult to observe blood vessels in broad areas including the burn injuries.

### 3. Speculation

We studied the effects of the new material CS (the silk carbonized body) to burn injuries and confirmed that symptom of edema could be lessened and wounds could be rapidly cured by the new material.

We think that the effects of the CS are brought by the following three reasons.

Firstly, the CS makes secretions easily come out from a surface of a body. As shown in FIG. 10, in the CS(+) group, the thickness of the skins immediately under the burn injuries were significantly thinner than that of the normal skins. Namely, it means that the symptom of the edema immediately under the burn injuries were apparently reduced in the CS(+) group. Since the petrolatum used as a base agent was not soluble in water, it was impossible for moisture to move to surfaces of the wounds by osmotic pressure. We think that the CS has a unique characteristic of absorbing secretions.

Secondly, the CS has a characteristic of migration of inflammatory cells. Generally, a process of curing a wound is constituted by three stages: inflammation; granulation; and reconstruction. Depth of a burn injury progresses for 48 hours from receiving the wound, so the burn injuries on the third days of the Example were in the inflammation stage or an early stage of the wound. In the inflammation stage, neutrophil and monocyte cells phagocytize damaged cells and secrete physiologically active substances, e.g., cytokine. Neutrophil mainly act in the early stage of the cure process, and they disappear in a short time after a lapse of three days, so phagocytizing and decomposing foreign substances by neutrophil is important for normal progress of the cure process. Time course of cell kinetic after receiving a wound will be studied in detail, but the above described experiments teach that the CS makes the cure process, which is mainly performed by neutrophil, progress smoothly. Since the samples shown in FIG. 12, in which crusta was peeled from epidermis, were in the CS(+) group only, the CS accelerated the cure process, we think.

Thirdly, the CS restricts vascular permeability, so that generation of edemas can be directly restricted. Inflammation attractants are released from cells in tissues by physical or chemical irritations, so that hyperlucency of capillary walls is caused and edemas of tissues are generated. Histamine, bradykinin, vascular endothelial growth factor (VEGF), etc. are know as inflammation factors. Histamine has a vasodilator property and a capillary vessel hyperlucency property; VEGF is a mediator for forming new blood vessels and increasing capillary vessel permeability. Edemas are formed when amount of liquid departed from capillary vessels is greater than flow volume of lymph draining tissues of that part, but expansion of blood vessels and lymph vessels, which are caused by drainage of tissue liquid, were not observed in the CS(+) group. As shown in FIG. 13, no blood vessels were observed in broad areas including the burn injuries. Therefore, we think that the CS has the characteristic of directly restricting functions of vascular permeability substances, e.g., VEGF.

1. A wound-covering material made of a silk carbonized body, which is produced by carbonizing a silk material, wherein a large number of microfine holes are formed in a surface of the carbonized silk body.

2. The wound-covering material according to claim 1, wherein the silk material is carbonized at temperature of 500-1000° C.

3. The wound-covering material according to claim 1, wherein the silk carbonized body includes 5-35 wt % of nitrogen elements.

4. The wound-covering material according to claim 1, wherein the microfine holes are formed by activation treatment.

5. The wound-covering material according to claim 2, wherein the microfine holes are formed by activation treatment.

6. The wound-covering material according to claim 3, wherein the microfine holes are formed by activation treatment.

7. The wound-covering material according to claim 1, wherein the silk carbonized body is formed like woven cloth, knitted fabric or unwoven cloth.

8. The wound-covering material according to claim 1, wherein the silk carbonized body is formed like yarns.

9. The wound-covering material according to claim 1, wherein the silk carbonized body is broken into shatters.

10. The wound-covering material according to claim 2, wherein the silk carbonized body is broken into shatters.

11. The wound-covering material according to claim 3, wherein the silk carbonized body is broken into shatters.

12. The wound-covering material according to claim 4, wherein the silk carbonized body is broken into shatters.

13. The wound-covering material according to claim 5, wherein the silk carbonized body is broken into shatters.

14. The wound-covering material according to claim 6, wherein the silk carbonized body is broken into shatters.

15. The wound-covering material according to claim 1, wherein said wound-covering material includes a wound therapeutic drug.
